(19) United States
(12) Patent Application Publication
(10) Pub. No.: US 2023/0264193 A1
(43) Pub. Date: Aug. 24, 2023

(54) SYSTEM FOR ANALYSIS

(71) Applicant: miDiagnostics NV, Heverlee (BE)

(72) Inventors: Christopher ADAMS, Heverlee (BE); David MIKAELIAN, Heverlee (BE); Klaas DE GREVE, Heverlee (BE)

(21) Appl. No.: 18/040,257

(22) PCT Filed: Aug. 13, 2021
(86) PCT No.: PCT/EP2021/072630

§ 371 Date: Feb. 1, 2023

(30) Foreign Application Priority Data
Aug. 14, 2020 (EP) 20191196.3

(51) Int. Cl. B01L 3/00; G01N 33/80

(52) U.S. Cl. CPC B01L 3/502715; G01N 33/80; B01L 2300/0883; B01L 2400/0406; B01L 2200/027; B01L 2300/0816; B01L 2300/0887; B01L 2200/04; B01L 2200/16; B01L 2300/0654

(57) ABSTRACT

There is provided a microfluidic device comprising a disposable microfluidic test card for capillary driven liquid sample processing, the disposable microfluidic test card comprising a sample inlet, arranged for receiving liquid sample at the microfluidic test card, at least one test reagent reservoir arranged for holding of test reagent, an analysis zone for analysis of liquid sample components, and a microfluidic sample processing zone arranged in fluidic connection with the sample inlet and the at least one test reagent reservoir, for receiving of liquid sample and test reagent, respectively, therefrom, the microfluidic sample processing zone being further arranged for metering and providing a predetermined volume of liquid sample, mixing or contacting of the predetermined volume of liquid sample with test reagent, allowing processing of liquid sample mixed or contacted with test reagent, and fluidic connection with the analysis zone for providing processed liquid sample to the analysis zone, wherein the analysis zone is arranged for presenting processed liquid sample to a reader.

### TECHNICAL FIELD OF INVENTION

The present invention relates to a microfluidic device, a system comprising the microfluidic device, and a method for performing liquid sample processing and analysis.

### TECHNICAL BACKGROUND

Accurate and precise diagnostic tests are an essential part of an effective and efficient healthcare system. Because achieving accuracy and precision often requires laboratory-scale equipment, a majority of diagnostic tests used in the medical practice today are performed in centralized laboratory settings, negatively impacting the total cost, time-to-result, and accessibility of diagnostic testing. Concepts to enable diagnostic testing at the point-of-care have been proposed, but at the cost of a reduced ability to manipulate a clinical sample in a workflow that ensures accuracy and precision.

There is, thus, a need for diagnostic testing solutions that are low-cost, easy-to-use and accessible. The WHO sexually transmitted diseases diagnostic initiative has published the ASSURED benchmark to assess whether a diagnostic solution addresses global needs. According to this benchmark, the solution needs to be: Affordable, Sensitive, Specific, User-friendly, Robust, Equipment-free, and Deliverable to end-users.

Multiple point-of-care solutions have been proposed for other types of diagnostic testing and some technologies have been successfully marketed, but these are still far removed from the simplicity and convenience of a glucose test allowing glucose monitoring in a drop of blood. Rapid diagnostic tests (RDTs) are one of the simplest forms of point-of-care diagnostic tests, typically consisting of a nitrocellulose wick coated in specific locations with reagents. Fluid driving occurs by capillary wicking of aqueous liquids in the nitrocellulose strips and diagnostic read-out occurs via the detection of colored bands either by the human eye, a ubiquitous device such as a smartphone, or a dedicated reader device. Because of their simplicity, RDTs may be affordable and often equipment-free, but often fail to meet the other requirements in the ASSURED criteria. Sensitivity and specificity are often suboptimal since one cannot perform extensive quality controls as one would in a laboratory setting.

At the more complex end of the spectrum, point-of-care solutions for molecular testing, consisting of more complex cartridges and instruments, are available. These systems achieve more compact dimensions than their central laboratory-based counterparts by miniaturizing the workflow into one-time-use cartridges that are actuated in various ways by an instrument. They are easier to use because the reagent delivery is built into a disposable such that the user only needs to apply a sample and run the appropriate program associated to the desired test-cartridge. The need to provide mechanical, thermal and optical interfaces between instrument and cartridge limits the degree to which the instruments can be miniaturized and implies a cost which is prohibitive for many point-of-care settings. The cost is driven by the initial investment required for the instrument, cost of the consumables, maintenance, required infrastructure, operator time, etc.

### SUMMARY OF INVENTION

An object of the present invention is to mitigate, alleviate or eliminate one or more of the above-identified deficiencies in the art and disadvantages singly or in any combination and solve at least one above-mentioned problem. Another object of the present invention is to provide an efficient or improved microfluidic device, for example for analysis of liquid sample, such as blood sample.

According a first aspect of the present inventive concept there is provided a microfluidic device comprising a disposable microfluidic test card for capillary driven liquid sample processing, the disposable microfluidic test card comprising a sample inlet, arranged for receiving liquid sample at the microfluidic test card, at least one test reagent reservoir arranged for holding of test reagent, an analysis zone for analysis of liquid sample components, and a microfluidic sample processing zone arranged in fluidic connection with the sample inlet and the at least one test reagent reservoir, for receiving of liquid sample and test reagent, respectively, therefrom, the microfluidic sample processing zone being further arranged for metering and providing a predetermined volume of liquid sample, mixing or contacting of the predetermined volume of liquid sample with test reagent, allowing processing of liquid sample mixed or contacted with test reagent, and fluidic connection with the analysis zone for providing processed liquid sample to the analysis zone, wherein the analysis zone is arranged for presenting processed liquid sample to a reader.

According to a second aspect of the present inventive concept, there is provided a system comprising the microfluidic device according to the first aspect

According to a further aspect of the present inventive concept, there is provided a method for performing liquid sample processing and analysis on a microfluidic system comprising a disposable microfluidic test card including a microfluidic sample processing zone. The method comprising: receiving liquid sample to the microfluidic test card; propagating by capillary action received liquid sample to the microfluidic sample processing zone; performing, as timed events, in the microfluidic sample processing zone: metering a predetermined volume of propagated liquid sample; isolating the predetermined volume of propagated liquid sample from remaining propagated liquid sample, thereby providing an isolated liquid sample having a predetermined volume; mixing or contacting the isolated liquid sample with a test reagent; processing the isolated liquid sample mixed or contacted with the test reagent, thereby obtaining processed liquid sample; and performing analysis of the processed liquid sample on the microfluidic test card.

A further scope of applicability of the present disclosure will become apparent from the detailed description given below. However, it should be understood that the detailed description and specific examples, while indicating preferred variants of the present inventive concept, are given by way of illustration only, since various changes and modifications within the scope of the inventive concept will become apparent to those skilled in the art from this detailed description.

Hence, it is to be understood that the inventive concepts are not limited to the particular steps of the methods described or component parts of the systems described as such method and system may vary. It is also to be understood that the terminology used herein is for purpose of describing particular embodiments only and is not intended to be limiting. It must be noted that, as used in the specification and the appended claim, the articles “a”, “an”, “the”, and “said” are intended to mean that there are one or more of the elements unless the context clearly dictates otherwise. Thus, for example, reference to “a unit” or “the unit” may include several devices, and the like. Furthermore, the words “comprising”, “including”, “containing” and similar wordings do not exclude other elements or steps.

### BRIEF DESCRIPTION OF THE DRAWINGS

The above and other aspects of the present inventive concept will now be described in more detail, with reference to appended drawings showing variants of the invention. The figures should not be considered limiting the invention to the specific variant; instead they are used for explaining and understanding the inventive concept.

As illustrated in the figures, sizes of components, layers or distances may be exaggerated for illustrative purposes and, thus, are provided to illustrate the general structures of variants of the present inventive concept. Like reference numerals refer to like elements throughout.

FIG. 1 schematically illustrates an aspect of the present inventive concept.

FIG. 2 illustrates a microfluidic device according to embodiments.

FIG. 3 illustrates a microfluidic device according to embodiments.

FIGS. 4 (a) and (b) illustrates a microfluidic sample processing zone according to embodiments.

FIG. 5 schematically illustrates a fluidic connection according to embodiments.

FIG. 6 schematically illustrates sample metering and/or a channel system according to embodiments.

FIG. 7 illustrates a system according to a second concept and/or embodiments.

FIG. 8 illustrates experimental results.

### DETAILED DESCRIPTION

With the present inventive concept, there is provided a technology that miniaturizes and simplifies a complete sample liquid analysis workflow. A series of operations may be executed autonomously in a compact disposable microfluidic test card without need of skilled professionals or use of using laboratory equipment. This has been enabled by precisely engineering capillary forces in fluidic microchip structures such that a sequence of steps is performed without requiring further human intervention and/or additional instrumentation or actuation to perform the operations. Further, for example, when combined with lens-free computational microscopy and/or computer vision techniques, these autonomously driven microfluidic systems may be a test card solution to enable desirable point-of-care diagnostics.

Fluidic operations may be enabled in and controlled by capillary forces that are used to propel liquids and to control operations such as valving, metering, incubating, and performing conditional operations.

The present inventive concept will now be described more fully hereinafter with reference to the accompanying drawings, in which currently preferred variants of the inventive concept are shown. This inventive concept may, however, be implemented in many different forms and should not be construed as limited to the variants set forth herein; rather, these variants are provided for thoroughness and completeness, and fully convey the scope of the present inventive concept to the skilled person.

It is to be understood that at least channels of the device may be capillary channels. A capillary channel may be considered as a channel capable of providing a capillary-driven flow of a liquid. It is also to be understood that other channels of the system may be capillary channels and/or other types of channels depending on the specific implementation of the present inventive concept.

In the following, fluid is described as flowing through channels and reaching certain positions at different times within the microfluidic system. Flow rates of these flows may be controlled in different manners in order for the fluid to reach the positions at the described times. A capillary-driven flow of a fluid requires one or more contacting surfaces that the fluid can wet. For example, surfaces comprising glass or silica may be used for capillary-driven flows of aqueous liquids. Further, for example, suitable polymers with hydrophilic properties, either inherent to the polymer or by modification, including for example chemical modification or coating, may promote or enhance capillary driven flows.

The flows may be controlled, for example, by adapting the length of the channels and/or by adapting the flow resistances of the channels. The flow resistance of a channel may be controlled by adapting a cross-sectional area of the channel and/or the length of the channel. The flow resistance of a channel may further be dependent on properties of the liquid, e.g. its dynamic viscosity. Additionally, or alternatively, the flow rate may be adapted by using flow resistors.

To provide desired capillary forces, dimensions of flow channels may be selected dependent on, for example, the properties of the liquid and/or material and/or properties of walls of the channels.

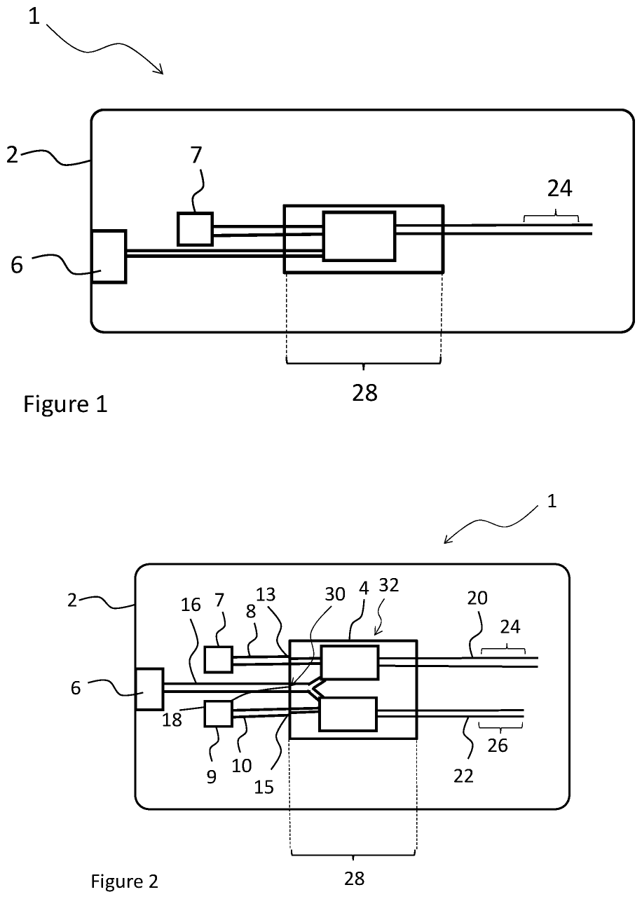

With reference to FIG. 1, a first aspect of the present inventive concept will now be discussed. A microfluidic device 1 is illustrated in FIG. 1. The device comprises a disposable microfluidic test card 2 for capillary driven liquid sample processing. The disposable microfluidic test card 2 comprises a sample inlet 6, arranged for receiving liquid sample (not illustrated) at the microfluidic test card 2, at least one test reagent reservoir 7 arranged for holding of test reagent, an analysis zone 24 for analysis of liquid sample components, and a microfluidic sample processing zone 28 arranged in fluidic connection with the sample inlet 6 and the at least one test reagent reservoir 7, for receiving of liquid sample and test reagent, respectively, therefrom, the microfluidic sample processing zone 28 being further arranged for metering and providing a predetermined volume of liquid sample, mixing or contacting of the predetermined volume of liquid sample with test reagent, allowing processing of liquid sample mixed or contacted with test reagent, and fluidic connection with the analysis zone 24 for providing processed liquid sample to the analysis zone 24, wherein the analysis zone 24 is arranged for presenting processed liquid sample to a reader (not illustrated).

The microfluidic device may be for analysis of liquid sample.

The disposable microfluidic test card may further comprises a microfluidic chip comprising the microfluidic sample processing zone.
Thereby, the chip and the microfluidic sample processing zone may be manufactured separate from the test card. Further, one test card or one type of test card may be used with differently designed chips or microfluidic sample processing zone.

An example of the microfluidic device 1 will now be described and discussed with reference to FIG. 2. The disposable microfluidic test card 2 may further comprise at least one pre-processing test reagent channel 8, in the illustrated example there are two pre-processing test reagent channels 8, 10, arranged for capillary drive of test reagent from the at least one test reagent reservoir 7, in the illustrated example there are two test reagent reservoirs 7, 9, to the microfluidic sample processing zone 28, at least one pre-processing sample channel 16 arranged for liquid communication with the sample inlet 6, and arranged for capillary drive of liquid sample to the microfluidic sample processing zone 28, at least one sample analysis channel 20, in the illustrated example there are two sample analysis channels 20, 22, for receiving processed liquid sample from the microfluidic sample processing zone 28 and for analysis of the processed liquid sample. Wherein the microfluidic sample processing zone may comprise a processing channel system 32 comprising at least one liquid sample entrance 30, arranged for receiving liquid sample from the at least one pre-processing sample channel 16 at least one test reagent entrance 13, 15, arranged for receiving test reagent from the at least one pre-processing test reagent channel 8, 10, at least one sample metering capillary channel having a predetermined volume, and arranged for providing the predetermined volume of received liquid sample to at least one processing channel, and the at least one processing channel, arranged for contacting, in the at least one processing channel, the test reagent and the predetermined volume of liquid sample, thereby allowing processing of liquid sample, and further arranged for capillary drive of processed liquid sample to the at least one sample analysis channel 20, 22 for analysis. The sample analysis channels 20, 22, as illustrated in FIG. 2, are associated with sample analysis zone 24 and sample analysis zone 26, respectively, arranged for presenting processed liquid sample to a reader. The disposable microfluidic test card 2 as illustrated may, optionally, further comprises a microfluidic chip 4 comprising the microfluidic sample processing zone 28.
Further, the at least one pre-processing sample channel 16 may have a sample liquid outlet 18 for presenting the liquid sample to the sample processing zone 28.

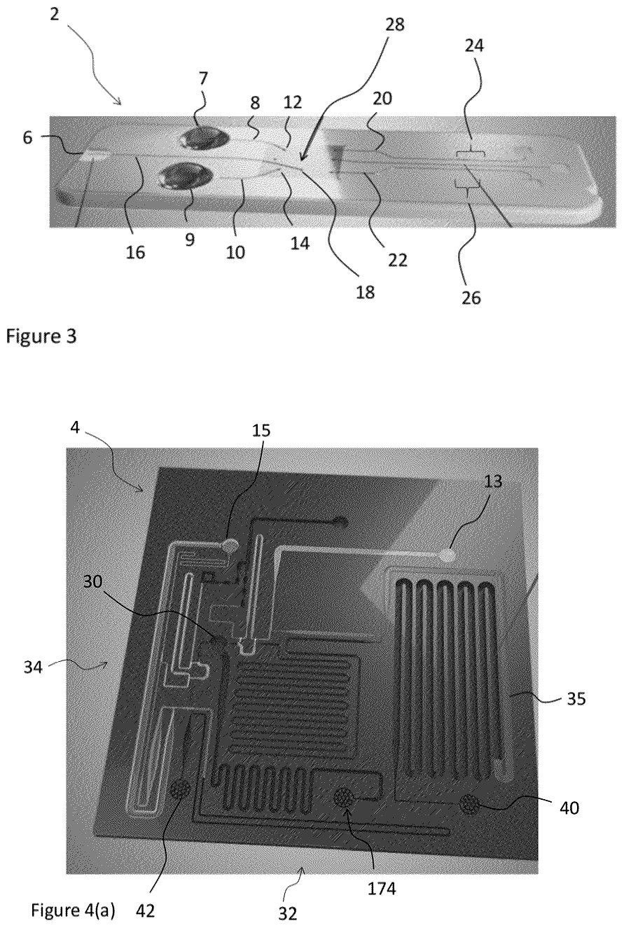

With reference to FIG. 3, the disposable microfluidic test card 2 according to an example and embodiment is illustrated. With the illustrated embodiment, the disposable microfluidic test card 2 further comprises a microfluidic chip comprising the microfluidic sample processing zone 28. The chip and sample processing zone 28 is visualised, in FIG. 3, as a darker grey area underneath a surface of the microfluidic test card 2 in an attempt to improve clarity. The microfluidic test card 2 comprises a sample inlet 6 (in this example positioned at an edge of the microfluidic test card 2, although it alternatively may be positioned otherwise) configured for receiving sample liquid at the test card; at least one test reagent reservoir 7, 9 arranged for holding of test reagent; at least one, for example as illustrated a first and a second, pre-processing test reagent channels 8, 10 having first and second test reagent outlets 12, 14, respectively, arranged for capillary drive of test reagent from the at least one test reagent reservoir 7, 9 to the microfluidic sample processing zone 28, or to the chip; at least one pre-processing sample channel 16 arranged for liquid communication with the sample inlet 6 for receiving liquid sample therefrom, and arranged for capillary drive of liquid sample to the microfluidic sample processing zone 28, the at least one pre-processing sample channel 16 may have a sample liquid outlet 18 for presenting the liquid sample to the sample processing zone 28 as illustrated; at least one, for example as illustrated, a first and a second processed sample analysis channel 20, 22 for receiving processed liquid sample from the microfluidic sample processing zone 28. The at least one, for example the first and the second processed sample analysis channels 20, 22, comprising an analysis zone 24, for example the illustrated first and second analysis zone 24, 26, respectively, for analysing the processed liquid sample.

Although first and second pre-processing test reagent channels 8, 10 having first and second test reagent outlets 12, 14, and the first and the second processed sample analysis channels 20, 22 comprising a first and second analysis zone 24, 26 are illustrated, it shall be appreciated that a device and a system may have, for example, only one of each channels present and the microfluidic chip and reader may suitably be adapted accordingly.

The microfluidic test card 2 may allow for reagent and sample introduction, integration of additional components such as capillary wicks and imaging zones, and may provide a more convenient form factor for manual handling. The microfluidic test card 2 may be built up out of several patterned layers that are laminated onto each other starting from eg. an injection-molded baseplate. For integration of the microfluidic chip into/onto the microfluidic test card 2 fluids need to transition from microfluidic test card 2 into the microfluidic chip 4 and vice versa by capillary wicking/forces. This may be achieved through design of the microfluidic chip 4 outlets and/or inlets/entrances, which feature wicking features to ensure rapid wicking to the surface of the microfluidic chip 4, and through design of features of foil laminates. Fluidic transitions from fluidic channels in the microfluidic test card 2 to capillary wicks/channels that act as waste reservoirs may be engineered to ensure adequately low failure rates.

The microfluidic sample processing zone 28 or the microfluidic chip, may have precisely engineered microfluidic channel geometries and surface properties. Fluids may propagate by capillary wicking but may be stopped by a geometric feature referred to as a trigger valve and, be triggered to continue again beyond the valve. To achieve reliable operation, the microfluidic sample processing zone 28 or the microfluidic chip may be constructed using a process that generates closed microfluidic channels by capping a first wafer that contains chips with etched channels with a cover wafer. The process for the bottom wafer may implement two etch depths, while the top wafer may have recesses in addition to fluidic access holes, resulting in three levels of microfluidics that may be combined to achieve the required component and system performance. Control over geometry of microfluidic channels and surface properties, may be achieved by leveraging silicon chip manufacturing techniques. Geometric control over both the horizontal and vertical dimensions may be achieved by relying on lithography, such as e.g. deep UV lithography or i-line lithography, and silicon deep reactive ion etch techniques, respectively. A well-defined contact angle may be achieved by coating the silicon surface, including in buried channels, with a surface-assembled monolayer that covalently bonds to the microfluidic sample processing zone 28 or the microfluidic chip surface from vapor phase. The microfluidic chip manufacturing approach may comply with silicon foundry processes such that microfluidic chip 4 manufacturing may flexibly be performed at a manufacturing site of choice.

The operation of a trigger valve, may rely on availability of three fluidic levels to achieve reliable operation in terms of their ability to hold without leaking, may be reliably triggered, and may operate without forming undesired bubbles which might otherwise impede the operation of the system. An ability to program a complex sequence of fluidic operations may enable the integration of a full sample workflow in an autonomously operating microfluidic sample processing zone or silicon microfluidic chip, such as the microfluidic sample processing zone 28 or the microfluidic chip.

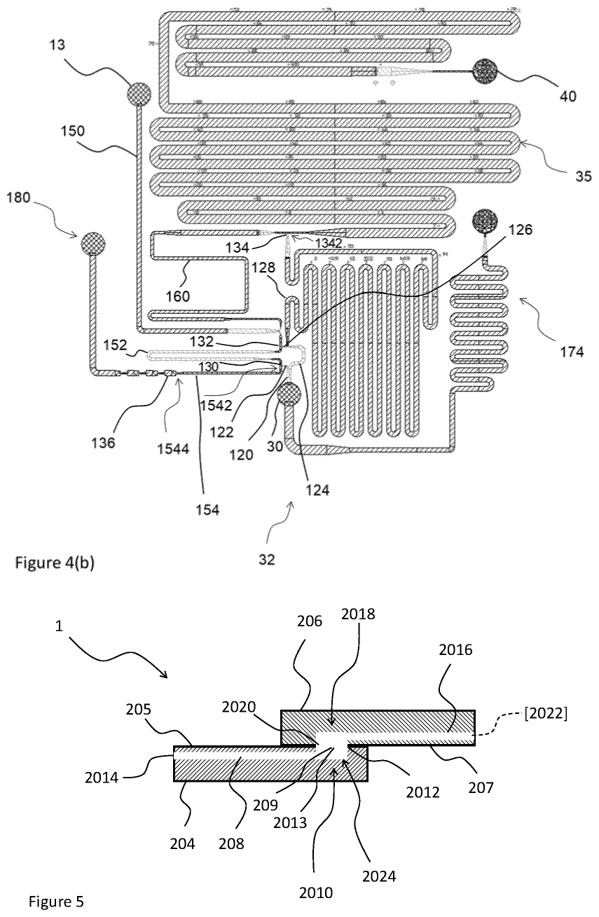

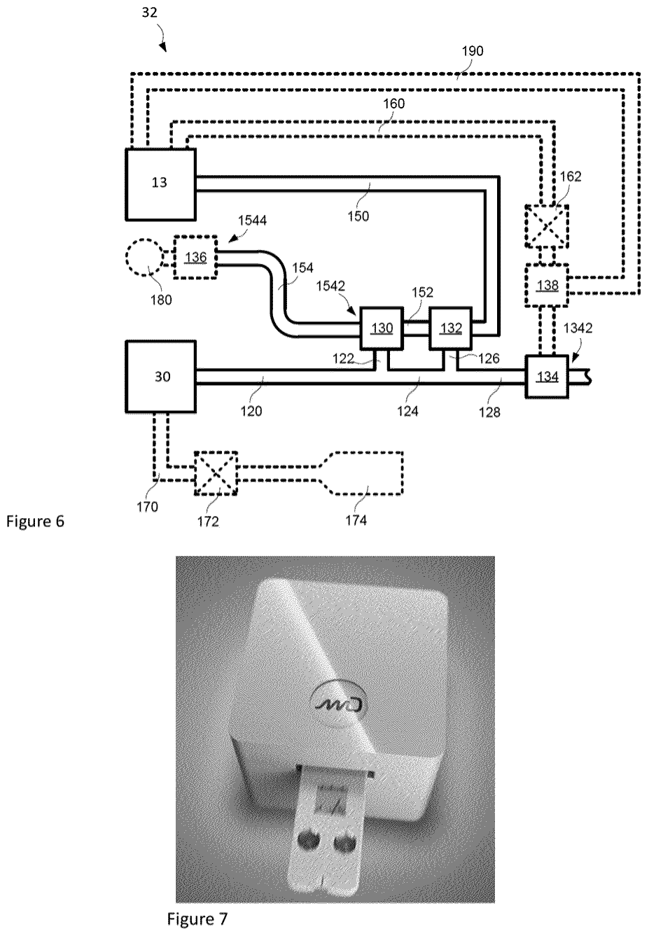

Combining a trigger valve with a high resistance fluidic channel, may result in a programmable or predeterminable delay function. For example by tuning channel fluidic resistance and relying on the coordination of several competing menisci, fluids may be driven in a complex sequence of steps not usually considered possible in a capillary-driven system, including reversal of fluid motion. By tuning channel dimensions, specific operations may be made conditional, as discussed below. The microfluidic sample processing zone 28 or the microfluidic chip design may accept three fluids: for example a blood sample, an aqueous dilution test reagent solution, and an aqueous red cell lysis test reagent solution. The microfluidic sample processing zone 28 or the microfluidic chip according to examples may execute a sequence of operations on the sample, some of which may be gated by reagents. First, the sample may arrive at the microfluidic sample processing zone 28 or the microfluidic chip sample entrance and be diverted into three simultaneous streams. Two of the streams may be designed to meter a specific volume of the sample, for example 100-1000 nL, eg. 600 nL, and 5-50, eg. 10 nL respectively, and the third stream may remove excess applied sample. In a subsequent step, the metered volumes may be isolated from trailing sample liquid plugs by replacing the upstream part of the fluid plug with test reagent solution using a design similar to, or of the type, illustrated in FIGS. 4 and 6. Subsequently, the eg. 10 nL metered volume of sample may then be diluted by a factor of eg. 400 by a dilution test reagent, while the eg. 600 nL metered volume may be mixed with a lysis test reagent, eg. in a ratio 1:5.

The microfluidic chip and the microfluidic test card 2 may be arranged with their respective channels oriented in different planes, eg. parallel planes, for example the microfluidic chip and the microfluidic test card 2 may be arranged one on top of the other. Thereby it shall be realized that liquid communication between eg. first and second pre-processing test reagent channels, and channels of the chip may be realized via eg. channels or openings having a direction or flow direction orthogonal to the plane of the microfluidic chip and the microfluidic test card 2.

For example, embodiments described with reference to FIGS. 1, 2, and 3 may further be described with reference to FIG. 5. FIG. 5 illustrates a microfluidic arrangement 1 for capillary driven fluidic connection between capillary flow channels, such as between pre-process sample 16, 2014 or pre-process test reagent channels 8, 10, 2014 and connected/corresponding microfluidic channels or systems. The microfluidic arrangement 1 comprises a first microfluidic system, or the microfluidic test card 2 comprising a first surface, and a first capillary flow channel 208, wherein the first capillary flow channel 208 has an elongation in a first plane, and the first surface comprises an outlet opening 209, eg. the sample/test reagent outlets, in a plane different from the first plane, the outlet opening defining an outlet area in the first surface and being adapted to allow fluidic communication with the first capillary flow channel thereby forming a flow outlet of the first capillary flow channel, and a second microfluidic system comprising a second surface and a second capillary flow channel, wherein the second capillary flow channel has an elongation in a second plane parallel to the first plane, and a portion of the second surface comprises an inlet opening in a plane different from the second plane, the inlet opening defining an inlet area in the second surface and being adapted to allow fluidic communication with the second capillary flow channel thereby forming a flow inlet of the second capillary flow channel, wherein the first microfluidic system and the second microfluidic system are arranged with the first and the second surfaces in contact such that the flow outlet and the flow inlet are interfaced, thereby allowing capillary driven fluidic connection between the first and the second capillary flow channels, wherein the outlet area overlaps at least a portion of the inlet area, said at least a portion of the inlet area overlapped by the outlet area being smaller than the outlet area. In analogy, the fluidic communication between exits of the microfluidic chip and the processed sample analysis channels may be fluidically connected as described with reference to FIG. 5.

The disposable microfluidic test card may be arranged for providing capillary driven flows of liquid through the channels of the disposable microfluidic test card.

The mixing or contacting of the predetermined volume of liquid sample with test reagent, and the allowing processing of liquid sample mixed or contacted with test reagent may be controlled in time, such as by use of capillary timing circuits and/or trigger valves.

The disposable microfluidic test card may further comprise or being connected to one or more of fluid outlets, collecting or waste reservoirs, and capillary pumps for providing capillary driven flows within or out of the disposable microfluidic test card, optionally, the disposable microfluidic test card may further comprise or being connected to vents for venting of gaseous medium from one or more channels of the disposable microfluidic test card.

The capillary pumps, when part of the device, may be selected from one or more of paper pump or elongated channel type of pumps.

The analysis zone may comprise a flow cell.

When the disposable microfluidic test card further comprises a microfluidic chip comprising the microfluidic sample processing zone, the disposable microfluidic test card may be manufactured from or comprises material selected from the group consisting of polymer, silica and glass, and combinations thereof.

The liquid sample may be blood or liquid derived from or comprising blood, the test reagent may be lysing buffer comprising lysing reagent for lysing of red blood cells present within the microfluidic sample processing zone, or dilution buffer for diluting the predetermined volume of liquid sample, and the at least one sample analysis channel may be for analysis comprising determining a measure of leukocytes, and/or determining a measure of red blood cells.

According to a second aspect of the present inventive concept, there is provided a system comprising the microfluidic device according to the first aspect.

The system may further comprise a reader comprising a sensor unit arranged for receiving a signal for processing and transmitting a processed signal to a computer, preferably the sensor unit may be selected from an optical unit, a Cmos camera, and a colorometry detection unit, and, optionally, a computer readeable medium configured to display information from the processed signal, and a storage/communication means. An example of such a system is illustrated in FIG. 7.

The sensor unit may sense and receive the signal indicative of presence of processed liquid sample in the analysis zone.

According to a further aspect of the present inventive concept, there is provided a method for performing liquid sample processing and analysis on a microfluidic system comprising a disposable microfluidic test card including a microfluidic sample processing zone. The method comprising: receiving liquid sample to the microfluidic test card; propagating by capillary action received liquid sample to the microfluidic sample processing zone; performing, as timed events, in the microfluidic sample processing zone: metering a predetermined volume of propagated liquid sample; isolating the predetermined volume of propagated liquid sample from remaining propagated liquid sample, thereby providing an isolated liquid sample having a predetermined volume; mixing or contacting the isolated liquid sample with a test reagent; processing the isolated liquid sample mixed or contacted with the test reagent, thereby obtaining processed liquid sample; and performing analysis of the processed liquid sample on the microfluidic test card.

Such predetermined sample volumes may be provided by means of an arrangement illustrated with reference to FIG. 6. The arrangement of FIG. 6 may be or be part of the microfluidic sample processing zone 28 or the processing channel system 32 although references in the discussion are made to one and the first processing channel system 32. FIG. 6 illustrates a first processing channel system 32 for providing a sample liquid (sample liquid not illustrated in FIG. 6) having a predetermined sample volume. The first processing channel system 32 is arranged in fluidic communication with sample liquid outlet 18 via the sample liquid entrance 30, thus arranged for receiving sample liquid. The first processing channel system 32 further comprises a first processing sample channel 120 connected to the sample liquid entrance 30. The first processing sample channel 120 branching off into a second processing sample channel 122 ending in a first valve 130, and into a third processing sample channel 124. The third processing sample channel 124 branching off into a fourth processing sample channel 126 ending in a second valve 132, and into a fifth processing sample channel 128 ending in a third valve 134, wherein the fifth processing sample channel 128 has a predetermined volume. The first valve 130, the second valve 132, and/or the third valve 134 may be trigger valves. A trigger valve may, in its closed state, stop a main liquid flow, and in its opened state, allow the main liquid flow to pass through the trigger valve. The trigger valve may be opened (i.e. changed to its opened state) by a secondary flow, and a combined flow of the main flow and the secondary flow may be allowed to flow through an output of the trigger valve. Such trigger valves may within the art be known as capillary trigger valves. The illustrated first microfluidic channel system 32 further is configured in fluidic communication with the first test reagent outlet 12 via test reagent entrance 13 arranged for receiving a first test reagent. The first test reagent entrance 13, thus, may be arranged for receiving the test reagent.
The first processing channel system 32 further comprises a first trigger channel 150 arranged to connect the first test reagent entrance 13 to the second valve 132. The microfluidic channel system 32 further comprises a second trigger channel 152 connecting the second valve 132 and the first valve 130.
The first processing channel system 32 further comprises an exit channel 154 having a first end 1542 and a second end 1544. The first end 1542 is connected to the first valve 130. The first processing sample channel 120 is arranged to draw sample liquid from the sample entrance 30 to fill the first, second, third, fourth, and fifth processing sample channels 120, 122, 124, 126, 128 by capillary action. The flows of sample liquid are stopped by the first valve 130, the second valve 132, and the third valve 134, as the valves are in their closed states.

The first trigger channel 150 is arranged to draw test reagent from the first test reagent entrance 13, by capillary action, to the exit channel 154 via a liquid path comprising the second trigger channel 152, and to open the second valve 132 and the first valve 130, whereby a further liquid path comprising the fourth processing sample channel 126, the third processing sample channel 124, and the second processing sample channel 122 is opened up. The opened further liquid path allows for sample present in the fourth processing sample channel 126, the third processing sample channel 124, and the second processing sample channel 122 to be replaced by test reagent from the first trigger channel 150 and flow into the exit channel 154 together with test reagent from the second trigger channel 152, thereby isolating a sample liquid present in the fifth processing sample channel 128 from adjacent sample liquid. The first processing sample channel 120 and/or the fifth processing sample channel 128 may be adapted, e.g. by adapting their respective geometries (e.g., cross-sectional dimensions and/or shapes), such that capillary forces (or capillary pressures) prevent sample liquid present in the first processing sample channel 120 and/or the fifth processing sample channel 128 to flow towards the exit channel 154. The second processing sample channel 122, the third processing sample channel 124, the fourth processing sample channel 126, the first trigger channel 150, the second trigger channel 152 and/or the exit channel 154 may be adapted, e.g. by adapting their respective geometries (e.g., cross-sectional dimensions and/or shapes), such that sample liquid present in the second processing sample channel 122, the third processing sample channel 124 and the fourth processing sample channel 126 may be replaced by test reagent from the first trigger channel 150 and to flow into exit channel 154 together with test reagent from the second trigger channel 152.
A volume of the isolated sample liquid corresponds to the volume of the fifth processing sample channel 128, thereby providing the sample liquid having the predetermined sample volume.

Thus, the present first processing channel system 32 enables provision of sample liquid having a predetermined volume. The sample liquid having the predetermined sample volume is isolated from adjacent sample liquid in the microfluidic channel system 32, without actively controlling the flows within the microfluidic channel system 32.
As shown in the example of FIG. 6, the first processing channel system 32 may further comprise a timing channel 160 connecting the test reagent entrance 13 and the third valve 134. The timing channel 160 may be arranged to draw, by capillary action, test reagent from the first test reagent entrance, and thereby from the first pre-processing test reagent channel 8, to an output 1342, which may be the second exit 40, of the third valve 134 and to open the third valve 134, whereby the isolated sample liquid present in the fifth channel may be allowed to flow through the output 1342 of the third valve 134 together with test reagent from the timing channel 160. The output 1342 of the third valve 134 may be an output of the microfluidic channel system 32 configured for direct fluidic communication with the first processed sample channel 20, or via a channel for processing of the sample liquid processing channel. The test reagent may be eg. lysing test reagent, for lysing of, for example, red blood cells, or it may be a dilution test reagent for dilution of sample liquid.
The first processing channel system 32 may further comprise a channel 190 connected to a valve 138.

Hence, the isolated sample liquid may be extracted from the microfluidic channel system 32. It may, e.g., be provided to the microfluidic test card for analysis and/or further treatment. For analysis, it may be advantageous to precisely meter the sample liquid to be analysed, which may be allowed by the present microfluidic channel system 32. The timing channel 160 may be configured to open the third valve 134 subsequent to the sample liquid present in the fifth processing sample channel 128 being isolated from adjacent sample liquid. The timing channel 160 may be further configured to open the third valve 134 subsequent to sample liquid and test reagent reaching the second end 1544 of the exit channel 154. As is shown in the example of FIG. 6, the timing channel 160 may comprise a first flow resistor 162. A flow resistance of the first flow resistor 162 may be selected to control the flow rate from the test reagent entrance 13 to the third valve 134 such that the third valve 134 may be opened subsequent to sample liquid in the fifth processing sample channel 128 being isolated from adjacent sample liquid. Additionally, the flow resistance of the first flow resistor 162 may be selected to control the flow rate from the test reagent entrance 13 to the third valve 134 such that the third valve 134 may be opened subsequent to sample liquid and test reagent reaching the second end 1544 of the exit channel 154.

Thus, a length of the timing channel 160 may be decreased, while still allowing for the third valve 134 to be opened subsequent to the sample liquid in the fifth processing sample channel 128 being isolated from adjacent sample liquid.

As is shown in the example of FIG. 6, the first processing channel system 32 may further comprise a capillary pump 174 arranged to empty the sample liquid entrance 30 and/or a thereto connected sample reservoir. The capillary pump 174 may be arranged to empty the sample liquid entrance 30 subsequent to the first, second, third, fourth, and fifth processing sample channels 120, 122, 124, 126, 128 being filled with sample liquid. The capillary pump 174 may be a paper pump and/or a microfluidic channel structure configured to draw liquid from the sample liquid entrance 30. During emptying of the sample liquid entrance 30 by the capillary pump 174, capillary pressures or capillary forces in the second processing sample channel 122, in the fourth processing sample channel 126, and in the fifth processing sample channel 128 may counteract drawing of sample liquid from the first processing sample channel 120, the second processing sample channel 122, the third processing sample channel 124, the fourth processing sample channel 126, and the fifth processing sample channel 128 in a direction towards the sample liquid entrance 30. The capillary pressures or capillary forces in the second processing sample channel 122, in the fourth processing sample channel 126, and in the fifth processing sample channel 128 may be higher than the capillary pressure or capillary force generated by the capillary pump 174, thereby avoiding emptying the second processing sample channel 122, the fourth processing sample channel 126, and the fifth processing sample channel 128.

The sample liquid entrance 30 may thereby receive sample liquid having a larger volume than a combined volume of the first, second, third, fourth, and fifth processing sample channel 120, 122, 124, 126, 128, thereby reducing a need to limit the volume of the sample liquid received by the sample liquid entrance 30. In case sample liquid is present in the sample liquid entrance 30 subsequent to filling the first, second, third, fourth, and fifth processing sample channel 120, 122, 124, 126, 128, additional sample liquid may be drawn by capillary action from the sample liquid entrance 30 upon opening the first, the second, and/or the third valves 130, 132, 134. Emptying the sample liquid entrance 30 from liquid subsequent to filling the first, second, third, fourth, and fifth processing sample channel 120, 122, 124, 126, 128, allows a capillary pressure or capillary force at an interface between sample liquid in the first processing sample channel 120 and the sample liquid entrance 30 to counteract drawing of sample liquid from the first processing sample channel 120 in a direction from the sample liquid entrance 30.

The capillary pump 174 may be connected to the sample liquid entrance 30 via a second flow resistor 172. A flow resistance of the second flow resistor 172 may be selected to control the flow rate from the sample liquid entrance 30 to the capillary pump 174 such that the sample liquid entrance 30 may be emptied subsequent to the first processing sample channel 120, the second processing sample channel 122, the third processing sample channel 124, the fourth processing sample channel 126, and the fifth processing sample channel 128 being filled with sample liquid. The capillary pump 174 may be connected to the sample reservoir via a pump capillary channel 170, and the pump capillary channel 170 may comprise the second flow resistor 172.
The microfluidic channel system 32 may further comprise a stop valve 136 connected to the second end 1544 of the exit channel 154.

The microfluidic channel system 32 may further comprise a vent 180 connected to the stop valve 136. The vent 180 may be arranged to allow gaseous communication between the stop valve 136 and surroundings of the first processing channel system 32 such that gas present in the exit channel 154 may be allowed to escape. Gas present in one or more of the first processing sample channel 120, the second processing sample channel 122, the third processing sample channel 124, the fourth processing sample channel 126, the first trigger channel 150, and the second trigger channel 152 may be allowed to escape through the vent 180 via the exit channel 154. Additionally, gas present in one or more of the first processing sample channel 120, the second processing sample channel 122, the third processing sample channel 124, the fourth processing sample channel 126, the fifth processing sample channel 128, the first trigger channel 150, and the second trigger channel 152 may be allowed to escape through the output 1342 of the third valve 134. Gas present in the channels may result in a build-up of gaseous pressure in the channels, which may act against the flow of liquid in the channels by capillary action. By allowing gas to escape, such build-up may be avoided, thereby allowing for an improved flow of the sample liquid and/or the test reagent.

With reference to FIG. 4(a), a microfluidic chip of the device 1 for analysis of sample liquid according to an embodiment will now be discussed. The illustrated embodiment comprises processing channel systems 32, 34 as discussed and illustrated with reference to FIG. 6. The device 1 according to the example may comprise two, the first and the second processing channel system 32 and 34, as illustrated in FIG. 4(a). To improve clarity FIG. 4(b) schematically illustrate a microfluidic chip 4 similar to the one discussed with reference to FIG. 4(a) with a single processing channel system 32 illustrated to improve clarity. The microfluidic chip 4 comprises a liquid sample entrance 30, configured for fluidic communication with the sample liquid outlet of the test card (not illustrated) and receiving sample liquid therefrom, a first processing channel system for processing sample liquid, configured for fluidic communication with the first test reagent outlet 12 and thereby configured to receive first test reagent from the first pre-processing test reagent channel, and further configured for fluidic communication with the sample liquid entrance 30, and thereby configured to receive sample liquid from the pre-processing sample liquid channel, and to allow contacting between sample liquid and first test reagent within the first processing channel system, and a second processing channel system 34 (not illustrated in FIG. 4(b)) for processing sample liquid, configured for fluidic communication with the second test reagent outlet 14 and thereby configured to receive second test reagent from the second pre-processing test reagent channel, and further configured for fluidic communication with the sample liquid entrance 30, and thereby configured to receive sample liquid from the pre-processing liquid sample channel, and to allow contacting between liquid sample and second test reagent within the second microfluidic channel system 34, wherein the first and the second processing channel system 32, 34 (only first illustrated in FIG. 4(b)) comprises first and second exits 40, 42 (only first illustrated in FIG. 4(b)), respectively, configured in fluidic connection with the first and second processed sample analysis channels 20, 22, respectively, of the microfluidic test card 2.

It shall be understood that the first and the second processing channel system 32, 34 may have one or more channels and/or components in common, but typically each have one individual microfluidic channel.

Further illustrated in FIGS. 4(a,b), which illustrations may further be understood from discussions concerning FIG. 6, and referring to first first processing channel system, although analogously or similarly may be referring to a second processing channel system, is a first processing sample channel 120 connected to the sample liquid entrance 30. The first processing sample channel 120 branching off into a second processing sample channel 122 ending in a first valve 130, and into a third processing sample channel 124. The third processing sample channel 124 branching off into a fourth processing sample channel 126 ending in a second valve 132, and into a fifth processing sample channel 128 ending in a third valve 134, wherein the fifth processing sample channel 128 has a predetermined volume. The first valve 130, the second valve 132, and/or the third valve 134 may be trigger valves. A trigger valve may, in its closed state, stop a main liquid flow, and in its opened state, allow the main liquid flow to pass through the trigger valve. The trigger valve may be opened (i.e. changed to its opened state) by a secondary flow, and a combined flow of the main flow and the secondary flow may be allowed to flow through an output of the trigger valve. Such trigger valves may within the art be known as capillary trigger valves. The illustrated first processing channel system 32 further is configured in fluidic communication with the first test reagent outlet 12 via test reagent entrance 13 arranged for receiving a first test reagent. The first test reagent entrance 13, thus, may be arranged for receiving the test reagent.

The first processing channel system 32 further comprises a first trigger channel 150 arranged to connect the first test reagent outlet 12 to the second valve 132. The first processing channel system 32 further comprises a second trigger channel 152 connecting the second valve 132 and the first valve 130.
The first processing channel system 32 further comprises an exit channel 154 having a first end 1542 and a second end 1544. The first end 1542 is connected to the first valve 130 and the second end is connected to a stop valve 136 that has a gaseous connection to a vent 180 arranged to allow gaseous communication with surroundings gaseous medium, eg. air. The first processing sample channel 120 is arranged to draw sample liquid from the sample entrance 30 to fill the first, second, third, fourth, and fifth processing sample channels 120, 122, 124, 126, 128 by capillary action. The flows of sample liquid are stopped by the first valve 130, the second valve 132, and the third valve 134, as the valves are in their closed states. One, more, or all of the valves may be capillary trigger valves.

The first trigger channel 150 is arranged to draw test reagent from the first test reagent entrance 13, by capillary action, to the exit channel 154 via a liquid path comprising the second trigger channel 152, and to open the second valve 132 and the first valve 130, whereby a further liquid path comprising the fourth processing sample channel 126, the third processing sample channel 124, and the second processing sample channel 122 is opened up. The opened further liquid path allows for sample present in the fourth processing sample channel 126, the third processing sample channel 124, and the second processing sample channel 122 to be replaced by test reagent from the first trigger channel 150 and flow into the exit channel 154 together with test reagent from the second trigger channel 152, thereby isolating a sample liquid present in the fifth processing sample channel 128 from adjacent sample liquid. The first processing sample channel 120 and/or the fifth processing sample channel 128 may be adapted, e.g. by adapting their respective geometries (e.g., cross-sectional dimensions and/or shapes), such that capillary forces (or capillary pressures) prevent sample liquid present in the first processing sample channel 120 and/or the fifth processing sample channel 128 to flow towards the exit channel 154. The second processing sample channel 122, the third processing sample channel 124, the fourth processing sample channel 126, the first trigger channel 150, the second trigger channel 152 and/or the exit channel 154 may be adapted, e.g. by adapting their respective geometries (e.g., cross-sectional dimensions and/or shapes), such that sample liquid present in the second processing sample channel 122, the third processing sample channel 124 and the fourth processing sample channel 126 may be replaced by test reagent from the first trigger channel 150 and to flow into exit channel 154 together with test reagent from the second trigger channel 152.
A volume of the isolated sample liquid corresponds to the volume of the fifth processing sample channel 128, thereby providing the sample liquid having the predetermined sample volume, such as for example 600 nl or 10 nl, to mention a few examples.

Thus, the present first processing channel system 32 enables provision of sample liquid having a predetermined volume. The sample liquid having the predetermined sample volume is isolated from adjacent sample liquid in the first processing channel system 32, without actively controlling the flows within the first processing channel system 32.

As shown in the example of FIG. 6, the first processing channel system 32 may further comprise a timing channel 160 connecting the test reagent entrance 13 and the third valve 134. The timing channel 160 may be arranged to draw, by capillary action, test reagent from the first test reagent entrance, and thereby from the first pre-processing test reagent channel 8, to an output 1342, which leads to second exit 40, of the third valve 134 and to open the third valve 134, whereby the isolated sample liquid present in the fifth channel may be allowed to flow through the output 1342 of the third valve 134 together with test reagent from the timing channel 160. The output 1342 of the third valve 134 may be an output for direct fluidic communication with the first processed sample channel 20, or as in the illustrated example via a channel 35 for processing of the sample liquid, eg. lysing or mixing etc. The test reagent may be eg. lysing test reagent, for lysing of, for example, red blood cells, or it may be a dilution test reagent for dilution of sample liquid. The system may be designed for suitable dilution of the sample by the test reagent when the sample is actuated from sample channel 128. In embodiments with two or more processing channel systems 32, 34, the microfluidic channel systems may be designed and functioning similar to the discussion above, and may alternatively be designed for different metered sample volumes, dilution processing times etc.

A capillary pump 174 may as exemplified be arranged to empty the sample inlet/reservoir/entrance 30, for example subsequent to the first, second, third, fourth, and fifth processing sample channels 120, 122, 124, 126, 128 being filled with sample liquid. Further illustrated is a vent 180 arranged to allow gaseous communication with surroundings gaseous medium, eg. air.

The device may be configured for providing capillary driven flows of liquid through channels. For example, the channels may have capillary dimensions and/or flows may be propagated assisted by capillary pumps or paper pumps, eg. pumps driven by capillary effects or wicking effects, such as paper pumps. Pressure-assisted capillary driven flows may used with embodiments.

The device may comprise capillary valves, for example capillary trigger valves, at suitable positions in liquid connection with channels of the device, for manipulating or controlling flows of the device.

The at least one pre-processing test reagent channel may further have at least one test reagent entrances fluidically connected to at least one test reagent reservoir, preferably blister type-of reservoirs.

The test card may be further configured to be in contact with an analyser, detector, or reader for detecting and analyzing the liquid sample and/or components of the sample liquid.

The liquid sample may be blood or derived from blood, and the first test reagent may be lysing test reagent for lysing of red blood cells, and the second test reagent may be dilution test reagent for diluting the blood sample.

The sample liquid may be blood or liquid derived from blood, and the first test reagent may be lysing buffer for lysing of red blood cells present within the first microfluidic channel system, and the second test reagent may be dilution buffer for diluting the blood sample present within the second microfluidic channel system.

It will be appreciated that the device, system, method, and embodiments thereof, may be used for blood analysis as discussed herein, but alternatively for other analysis or lab-on-a-chip applications, such as PCT-reactions. Any suitable application, wherein liquids and/or reagents etc. are to be manipulated as enabled by the present device and/or system are considered.

### Development and Testing of Device

Development and testing of an embodiment of the device, wherein the disposable microfluidic test card further comprises a microfluidic chip comprising the microfluidic sample processing zone, will be discussed below. The device may suitably be used with embodiments of the system.

A microfluidic chip was integrated into plastic microfluidic test card that allows e.g. for reagent/test reagent and sample introduction, integration of additional components such as capillary wicks and imaging zones, and provide a more convenient form factor for manual handling. The microfluidic test card was built up out of several patterned layers that were laminated onto each other starting from an injection-molded baseplate, as described herein. With integration of the microfluidic chip into the microfluidic test card fluids may transition from the microfluidic test card into the microfluidic chip and vice versa by capillary wicking. This was achieved through design of the microfluidic chip outlets and inlets/entrances, which feature wicking features to ensure rapid wicking to the surface of the microfluidic chip, and through design of the features in the foil laminates. Similarly, the transitions from the fluidic channels in the microfluidic test card to the capillary wicks that act as waste reservoirs have been engineered to ensure adequately low failure rates.

### Holographic Microscopy Development and Testing

The device discussed above with the system according to embodiments of the second aspect presents the sample to a computational or lens-free holographic microscope consisting of a laser diode and complementary metal oxide-semiconductor imager with a pixel size of 1.1 μm and having an array size and no additional optical components. The microfluidic test card was positioned just above the image sensor with the flow cell above the sensor surface, while the laser diode was positioned above the image sensor to ensure uniform illumination. The laser diode was operated stroboscopic mode with 2 μs pulses below lasing threshold (i.e. in spontaneous emission mode) to ensure a spectrum broad enough to prevent unwanted interference fringes due to unintended thickness variations of the microfluidic test card laminates. The imager captured holograms that were the result of interference between the partially coherent beam emitted by the laser diode, and the light scattered by cells and other particles in the flow cells at a frame rate of 21 frames per second, synchronized with the laser pulses.

Holograms were subsequently reconstructed into microscopic images.

### Evaluation Using Clinical Samples

### Performance of Device and System:

Experiments were performed using a device as described above, and the system comprising such a device and a reader, according to an embodiment of the second aspect.

Performance of the device and system described herein was evaluated using surplus blood draws obtained and tested the same day from the University Hospital of Leuven. For training the WBC CNN, pure cell fractions of neutrophils, eosinophils, monocytes and lymphocytes were prepared by magnetic bead-based isolation. The samples were aliquoted and run on a Sysmex XN-350 as reference device.

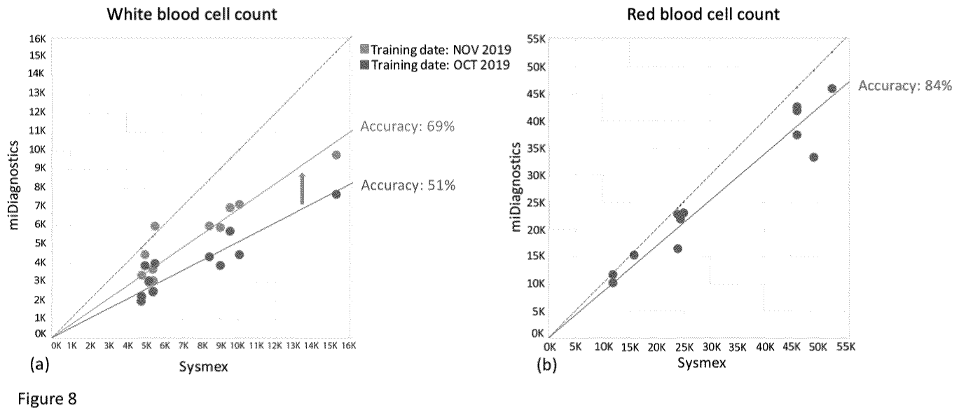

The achieved RBC and total WBC counts are shown for series of samples in FIGS. 8a and b, respectively, as a function of the counts achieved with the reference instrument.

Results demonstrate how the system using autonomously processing of a liquid sample and a lens-free in-flow microscopy system, can be combined to realize a point-of-care diagnostic solution for a complete blood count in a form factor and at a cost that is not otherwise conceivable. The microfluidic chip enables autonomously executing a number of operations on a sample and liquid reagent inputs without an electrical, optical or mechanical input from an instrument. The use of computational in-flow microscopy technique avoids the need for an optical system and its associated bulk, weight, complexity and cost.

### Microfluidic Test Card Fabrication

The channels of the microfluidic test card may be constructed by means of 42 μm-thick double-sided pressure sensitive adhesive (PSA) with the channel cut out of it. This PSA is sandwiched between two hydrophilized optically clear PET foils of 100 μm thick. The foils have a SiO₂ coating achieving a contact angle with deionized water of <20°. The arrangement is such that top and bottom of the fluidic channels are the PET foils with the hydrophilic surface exposed to the channel and the sidewalls the cut out edges from the PSA. Typical dimensions of channels width are 500 μm to 1 mm. The foil arrangement is supported by a baseplate acting as a structural support for the laminated foils as well as housing for the microfluidic chip and the capillary wicks which reside in recesses in the baseplate. The capillary wicks are blotting paper from Ahlstrom. The capillary microfluidic structures are created using a laminate of hydrophilic biocompatible foils. This foils assembly the attached to a backbone component that also contains the MICROFLUIDIC CHIP-Cell and fluidic drain mediums.

Most of the components are manufactured on site. The PMMA baseplate is moulded at a rapid prototyping house (Protomoulds). The cuts out of the channels out of the double side PSA and fluidic access holes in the other layers are manufactured by means of high precision laser cutting at dedicated laser machining workshops. The microfluidic chip is manufactured as described above.

The assembly was done under a flow hood to avoid particulate contaminations which are potentially detrimental for the fluidic flow or LFI imaging.

The different components are placed on top of each other by means of assembly jigs. These assembly jigs are made by laser cutting an acrylic plate to roughly a 10×10 cm plate and holes for inserting metal pins in certain locations. These metal pins have matching locations on the different layers. The bottom PSA, bottom hydrophilic foil, middle PSA (with channels cut out of them) and top hydrophilic foils are aligned in top of each other by these metal pins. The release liners on the PSA are removed prior to placing an additional layer on top. This arrangement is lightly pressed on to allow the different layers to stick together. All layers are handled by tweezers and only at the very edges. This to avoid excessive contact which might be detrimental to the hydrophilic layer or the LFI imaging.

The microfluidic chip is inserted into the baseplate recess by means of tweezer. The operator needs to pay attention to the orientation as the microfluidic chip is square (not a poka yoke insertion) and the fluidics channels need to be connected to the correct fluidic path in the microfluidic test card.

The paper wicks are cut to size by means of laser cutting. Just as the microfluidic chip they are inserted into the baseplate by means of tweezer.

The baseplate is then placed in the same jigs as used before and the four layers (cfr infra) are placed on top (with the final liner removed from the bottom PSA). The baseplate has the same alignment locations as the foils. The assembly is again lightly pressed to ensure that it is sticking together.

This assembly is subsequently passed through a roller laminator. The laminator has a certain compliance by means of silicone covered rollers. The microfluidic test cards are passed through it a single time. The lamination is there to fixate the layers and baseplate (with the microfluidic chip and paper wicks). After this lamination the test cards are ready for use.

### Holographic Microcopy Computational Approach

### Evaluation Using Clinical Samples

The CBC parameters of interest were total white blood cell count (WBC), the different WBC cell population counts (i.e. WBC differentiation) and the red blood cell count (RBC).

Accuracy in clinical testing paradigm was evaluated for WBC on venous whole blood samples covering a broad range of hematocrit (HCT) content. The samples were anonymized and surplus to requirement from blood draws on the same day obtained from the University Hospital of Leuven (UZ Leuven Gasthuisberg). The normal range for HCT in healthy persons is between 35% to 50%, for females between 35 and 45% and for males between 40 and 50%. The HCT values were sub-classified in 5 ranges, namely: 1) HCT up to 34% (low), 2) HCT from 35% to 39% (normal for females and low for males), 3) HCT from 40% to 44% (normal), 4) HCT from 45% to 50% (normal for males and high for females) and 5) HCT above 50% (high). Samples from two different donors per class were tested in 5 replicates (N=5 per sample, N=10 per class, N=50 in total).

Since different donors present a wide range of differences in their blood compositions and fluidic properties, it was also of interest to isolate the HCT parameter from other blood properties to determine the effect of HCT on accuracy and precision in the clinical testing. To this end, manipulated blood samples with 3 very distinct HCT values were made from whole blood from the same donor by centrifugation of two blood aliquots and transferring plasma from one fraction to the other to create low HCT (between 20% and 24%) and high HCT (between 50% and 54%) from the original normal HCT (35% to 45%) sample. This was done for two different donors, with each sample tested in 4 replicates (N=4 per sample, N=8 per class, N=24 in total). For a selection of 3 random whole blood samples, a high number of repeated tests were performed to evaluate the repeatability of WBC results by means of the imprecision metric the coefficient of variance (CV %). For our purposes we concluded at least 3 days of whole blood stability can be achieved when the plasma is substituted with Alsever's solution and the sample is stored in the fridge (2-8° C.). This extended shelf life is required to allow repeated testing over multiple days to obtain approx. 20-60 replicates of the same sample, since it is the aim to obtain 10 successful tests per sample to evaluate precision. The stabilization of whole blood was shown to have no adverse effect on the test performance (data not shown).

Accuracy and precision of RBC counts was evaluated on whole blood samples diluted with phosphate buffered saline (PBS) in the microfluidic chip at dilution rates from 200 fold to 800 fold to evaluate the impact of dilution ratio on the results.

Each blood sample was measured prior and after testing on Sysmex XN350 to obtain reference CBC values and to confirm sample integrity. For samples tested during a prolonged period (i.e. those for precision and repeatability testing), additional intermediate Sysmex measurement were undertaken.

After the initial sample introduction to the inlet port on the silicon microfluidic chip the first observations of the test were performed under the infra-red (IR) microscope to evaluate the microfluidic chip performance. Whole blood (6 μL) was dispensed into the blood inlet of the microfluidic test card with a pipette and the internal microfluidic chip volume metering was visualized by IR imaging, while excess whole blood was removed through directing the excess to a ‘waste’ channel integrated onto the microfluidic test card directly connected to an on-board paper pump integrated in the microfluidic test card. Following the precise cell metering volume step onboard the microfluidic chip 30 μL lysis test reagent was dispensed at the corresponding inlet to the microfluidic chip and the dilution, mixing and lysis was also followed by IR imaging.

When the sample was visualized at the outlet of the microfluidic chip, the microfluidic test card was removed from the IR microscope and slotted into the LFI reader where holograms/LFI images were generated and collected. LFI data was collected at high frame rate (21 frames per second (fps)). Holograms/LFI images were uploaded to the cloud based storage solution for processing.

1. A microfluidic device comprising
a disposable microfluidic test card for capillary driven liquid sample processing, the disposable microfluidic test card comprising
a sample inlet, arranged for receiving liquid sample at the microfluidic test card,
at least one test reagent reservoir arranged for holding of test reagent,
an analysis zone for analysis of liquid sample components, and
a microfluidic sample processing zone arranged in fluidic connection with the sample inlet and the at least one test reagent reservoir, for receiving of liquid sample and test reagent, respectively, therefrom, the microfluidic sample processing zone being further arranged for
metering and providing a predetermined volume of liquid sample,
mixing or contacting of the predetermined volume of liquid sample with test reagent,
allowing processing of liquid sample mixed or contacted with test reagent, and
fluidic connection with the analysis zone for providing processed liquid sample to the analysis zone,
wherein the analysis zone is arranged for presenting processed liquid sample to a reader.

2. The microfluidic device according to claim 1, wherein the disposable microfluidic test card further comprises a microfluidic chip comprising the microfluidic sample processing zone.

3. The microfluidic device according to claim 1, wherein the disposable microfluidic test card further comprises
at least one pre-processing test reagent channel arranged for capillary drive of test reagent from the at least one test reagent reservoir to the microfluidic sample processing zone,
at least one pre-processing sample channel arranged for liquid communication with the sample inlet, and arranged for capillary drive of liquid sample to the microfluidic sample processing zone,
at least one sample analysis channel for receiving processed liquid sample from the microfluidic sample processing zone and for analysis of the processed liquid sample,
and wherein
the microfluidic sample processing zone comprises
a processing channel system comprising
at least one liquid sample entrance, arranged for receiving liquid sample from the at least one pre-processing sample channel
at least one test reagent entrance, arranged for receiving test reagent from the at least one pre-processing test reagent channel,
at least one sample metering capillary channel having a predetermined volume, and arranged for providing the predetermined volume of received liquid sample to at least one processing channel, and
the at least one processing channel, arranged for contacting, in the at least one processing channel, the test reagent and the predetermined volume of liquid sample, thereby allowing processing of liquid sample, and further arranged for capillary drive of processed liquid sample to the at least one sample analysis channel for analysis.

4. The microfluidic device according to claim 1, wherein the disposable microfluidic test card is arranged for providing capillary driven flows of liquid through the channels of the disposable microfluidic test card.

5. The microfluidic device according to claim 1, wherein the mixing or contacting of the predetermined volume of liquid sample with test reagent, and the allowing processing of liquid sample mixed or contacted with test reagent is controlled in time, such as by use of capillary timing circuits and/or trigger valves.

6. The microfluidic device according to claim 1, wherein the disposable microfluidic test card further comprises or being connected to one or more of fluid outlets, collecting or waste reservoirs, and capillary pumps for driving of capillary driven flows within or out of the disposable microfluidic test card,
optionally, the disposable microfluidic test card further comprises or being connected to vents for venting of gaseous medium from one or more channels of the disposable microfluidic test card.

7. The microfluidic device according to claim 6, wherein the capillary pumps are selected from one or more of paper pump or elongated channel type of pumps.

8. The microfluidic device according to claim 1, wherein the analysis zone comprises a flow cell.

9. The microfluidic device according to claim 2, wherein the disposable microfluidic test card is manufactured from or comprises material selected from the group consisting of polymer, silica and glass, and combinations thereof.

10. The microfluidic device according to claim 1, wherein
the liquid sample is blood or liquid derived from or comprising blood,
the test reagent is
lysing buffer comprising lysing reagent for lysing of red blood cells present within the microfluidic sample processing zone, or
dilution buffer for diluting the predetermined volume of liquid sample, and
the at least one sample analysis channel is for analysis comprising
determining a measure of leukocytes, and/or
determining a measure of red blood cells.

11. A system comprising the microfluidic device according to claim 1.

12. The system according to claim 1, further comprising
a reader comprising a sensor unit arranged for receiving a signal for processing and transmitting a processed signal to a computer, preferably the sensor unit is selected from an optical unit, a CMOS camera, and a colorometry detection unit, and, optionally,
a computer readable medium configured to display information from the processed signal, and
a storage/communication means.

13. The system according to claim 12, wherein the sensor unit senses and receives the signal indicative of presence of processed liquid sample in the analysis zone.

14. A method for performing liquid sample processing and analysis on a microfluidic system comprising a disposable microfluidic test card including a microfluidic sample processing zone, the method comprising:
receiving liquid sample to the microfluidic test card;
propagating by capillary action received liquid sample to the microfluidic sample processing zone;
performing, as timed events, in the microfluidic sample processing zone:
metering a predetermined volume of propagated liquid sample;
isolating the predetermined volume of propagated liquid sample from remaining propagated liquid sample, thereby providing an isolated liquid sample having a predetermined volume;
mixing or contacting the isolated liquid sample with a test reagent;
processing the isolated liquid sample mixed or contacted with the test reagent, thereby obtaining processed liquid sample; and
performing analysis of the processed liquid sample on the microfluidic test card.
